(19) United States
(12) Patent Application Publication
(10) Pub. No.: US 2004/0109847 A1
(43) Pub. Date: Jun. 10, 2004

(54) Treatment and prophylaxis with 4-1BB-binding agents

(76) Inventors: Lieping Chen, Rochester, MN (US); Yang-Xin Fu, Chicago, IL (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/619,824

(22) Filed: Jul. 14, 2003

Related U.S. Application Data
(60) Provisional application No. 60/395,896, filed on Jul. 15, 2002.

(51) Int. Cl. A61K 38/21; A61K 39/395

(52) U.S. Cl. 424/85.4; 424/144.1

(57) ABSTRACT

Materials and methods for using 4-1BB agonists to treat or prevent autoimmune disorders, lymphoproliferative diseases, and allergies are provided.

### CROSS-REFERENCE TO RELATED APPLICATIONS

This application claims priority from U.S. Provisional Application Serial No. 60/395,896, filed Jul. 15, 2002.

### STATEMENT AS TO FEDERALLY SPONSORED RESEARCH

Funding for the work described herein was provided in part by the federal government, grant numbes CA79915 and HD37104. The federal government may have certain rights in the invention.

### TECHNICAL FIELD

This invention relates to materials and methods for treating and/or preventing autoimmune disorders, allergies, and lymphoproliferative diseases, and more particularly to materials and methods for using a 4-1BB agonist to treat and/or prevent autoimmune disorders, allergies, and lymphoproliferative diseases.

### BACKGROUND

Deletion of autoreactive lymphocytes in peripheral lymphoid tissues by apoptosis is an important mechanism for maintaining immune tolerance. This is demonstrated in MRL/lpr mice, which carry the lymphoproliferative (lpr) mutation of the Fas receptor gene on an autoimmune-prone background. These mice spontaneously develop lymphoproliferative disorders and lupus-like autoimmune diseases due to the lack of functional Fas/Fas ligand interactions. MRL/lpr mice also fail to properly deplete autoreactive lymphocytes by activation-induced cell death (AICD) (Theofilopoulos and Dixon (1981) Immunol. Rev. 55:179-216; and Cohen and Eisenberg (1991) Annu. Rev. Immunol. 9:243-269). The hallmark of lpr mutations is an accumulation of a unique population of thymic-derived CD4 and CD8 double negative T cells (DNTC) (TCR-α/β⁺) that aberrantly express the B220 (CD45) antigen (Wofsy et al. (1984) J. Immunol. 132:2686-2689; and Morse et al. (1982) J. Immunol. 129:2612-2615). Similarly, human autoimmune lymphoproliferative syndrome is due to defective Fas-induced apoptosis of activated lymphocytes (Sneller et al. (1992) J. Clin. Invest. 90:334-341; and Lenardo et al. (1999) Annu. Rev. Immunol. 17:221-253 (1999).

There are a limited number of immunotherapeutic approaches for treating lupus patients, whose morbidity and mortality rates remain relatively high. In murine autoimmune disease models, immunotherapeutic treatments have attempted to prevent T cell activation by administering blocking peptides, antibodies, and other agents that inhibit signaling through the TCR and costimulatory receptors (Kaliyaperumal et al (1999) J. Immunol. 162:5775-5783; Wofsy (1993) Immunol. Ser. 59:221-236; Mohan et al. (1995) J. Immunol. 154:1470-1480; Finck et al. (1994) Science 265:1225-1227; Kalled et al. (1998) J. Immunol. 160:2158-2165; and Liang et al. (2000) J. Immunol. 165:3436-3443). Still other approaches have exploited cytokine agonists and antagonists (Theofilopoulos and Lawson (1999) Ann. Rheum. Dis. 58 Suppl. 1:149-55; Kelley and Wuthrich, (1999) Semin. Nephrol. 19:57-66; and Lawson et al. (2000) J. Clin. Invest. 106:207-215). Some of the pitfalls of these therapies include the requirement for long-term treatment and their inability to deplete autoreactive lymphocytes and to reverse disease progression.

### SUMMARY

The invention is based on the discovery that, in a murine model of systemic lupus erythematosus (SLE), treatment with an agonistic antibody specific for the T cell costimulatory receptor 4-1BB resulted in decreased lymphadenopathy, decreased autoantibody production, decreased kidney disease, and prolonged survival. Beneficial effects were observed whether the animals were treated before or after onset of overt symptoms of disease. While the invention is not limited by any particular mechanism of action, the therapeutic and prophylactic effects of the 4-1BB-specific antibody apparently were mediated by increased apoptosis of CD4⁻, CD8⁻ double negative T cells (DNTC) and B cells. Thus the invention provides methods of using a 4-1BB agonist to deplete DNTC and/or autoreactive B cells for the treatment and/or prophylaxis of autoimmune diseases, hyper-proliferative diseases (e.g., lymphoproliferative diseases), and allergies. Moreover, the invention provides methods for inducing DNTC death.

In one aspect, the invention features a method for depleting double negative T cells in a subject. The method can include (a) identifying a subject as having, or at risk of having, an autoimmune disease, a lymphoproliferative disease, or an allergy; and (b) administering to the subject an effective amount of a 4-1BB agonist. The subject can be a human. The method can further include depleting autoreactive B cells in the subject, wherein the 4-1BB agonist is effective to deplete the autoreactive B cells. The 4-1BB agonist can be an antibody (e.g., a monoclonal antibody such as 2A) that binds to 4-1BB. The 4-1BB-binding agent can be 4-1BB ligand or a fragment thereof. The method can further include administering interferon-,y or a Gr-1 -binding agent (e.g., an antibody that binds to Gr-1) to the subject. The method can further include (c) monitoring the subject for symptoms of the autoimmune disease, lymphoproliferative disease, or allergy.

The autoimmune disease or the lymphoproliferative disease can be systemic lupus erythematosus or insulin-dependent diabetes mellitus. Alternatively, the autoimmune disease or the lymphoproliferative disease can be selected from the group consisting of an inflammatory bowel disease, a celiac disease, an autoimmune thyroid disease, Sjogren's Syndrome, autoimmune gastritis, pernicious anemia, autoimmune hepatitis, cutaneous autoimmune diseases, autoimmune dilated cardiomyopathy, myocarditis, myasthenia gravis, vasculitis, autoimmune diseases of the muscle, autoimmune diseases of the testis, autoimmune diseases of the ovary, and autoimmune diseases of the eye. The allergy can be to pollen antigens, fungal antigens, insect antigens, bacterial antigens, mammalian antigens, or insect venom antigens.

The administering can include delivering to the subject a nucleic acid containing a polynucleotide encoding the 4-1BB agonist, wherein the polynucleotide is operably linked to a transcriptional regulatory element. Alternatively, the administering can include (i) providing a cell from the subject; (ii) transfecting or transducing the cell, or a progeny of the cell, with a nucleic acid containing a polynucleotide encoding the 4-1BB-agonist, wherein the polynucleotide is operably linked to a transcriptional regulatory element; and (iii) administering the transfected or transduced cell, or a progeny of the transfected or transduced cell, to the subject.

In another aspect, the invention features a method for inducing death of a double negative T cell. The method can include contacting the double negative T cell with an effective amount of a 4-1BB agonist. The 4-1BB agonist can be an antibody (e.g., a monoclonal antibody such as 2A) that binds to 4-1BB. The 4-1BB agonist can be 4-1BB ligand or a fragment thereof. The method can further include inducing death of an autoreactive B cell, wherein the autoreactive B cell is contacted with the effective amount of the 4-1BB agonist.

The double negative T cell can be in vitro or in a subject (e.g., a human). The subject can have or be at risk for having an autoimmune disease, a lymphoproliferative disease, or an allergy. The autoimmune disease or the lymphoproliferative disease can be systemic lupus erythematosus or insulin-dependent diabetes mellitus. The autoimmune disease or the lymphoproliferative disease can be selected from the group consisting of an inflammatory bowel disease, a celiac disease, an autoimmune thyroid disease, Sjogren's Syndrome, autoimmune gastritis, pernicious anemia, autoimmune hepatitis, cutaneous autoimmune diseases, autoimmune dilated cardiomyopathy, myocarditis, myasthenia gravis, vasculitis, autoimmune diseases of the muscle, autoimmune diseases of the testis, autoimmune diseases of the ovary, and autoimmune diseases of the eye. The allergy can be to pollen antigens, fungal antigens, insect antigens, bacterial allergens, mammalian antigens, or insect venom antigens.

The contacting can include administering to the subject the 4-1BB agonist. The contacting can include administering to the subject a nucleic acid containing a polynucleotide encoding the 4-1BB agonist, wherein the polynucleotide is operably linked to a transcriptional regulatory element. Alternatively, the contacting can include (a) providing a cell from the subject; (b) transfecting or transducing the cell, or a progeny cell of the cell, with a nucleic acid containing a polynucleotide encoding the 4-1BB agonist, wherein the polynucleotide is operably linked to a transcriptional regulatory element; and (c) administering the transfected or transduced cell, or a progeny of the transfected or transduced cell, to the subject.

U.S. provisional Application Nos. 60/328,004 and 60/395,896 are incorporated herein by reference in their entirety.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1a, 1b, and 1c are scatter plots generated by flow cytometry of spleen cells from B6/lpr mice treated with an agonistic anti-4-1BB antibody (2A) or rat IgG control. The numbers represent cell percentages in each quadrant and are expressed as mean±SD (n=3). FIG. 1d is a graph showing total cell numbers of splenocytes, T cell subsets, B cells, and DNTC three weeks after the first treatment. Open columns, control; shaded columns, 2A-treated (n=3). FIG 1e is a dot plot showing levels of total IgG and anti-DNA IgG in sera from mice treated with 2A or IgG control as indicated. Open circles, control; filled circles, 2A-treated; *, P<0.05; **, P<0.01 by student's t test.

FIG. 2a is a graph showing number of palpable peripheral lymph nodes (pLNs) in control (open circles) and 2A-treated (filled circles) MRL/lpr mice (n=10). FIG. 2b is a graph of the weights of the spleen and pooled peripheral lymph nodes (pLN; including the inguinal, axillary, cervical lymph nodes), and mesenteric lymph nodes (mLN) in 2A-treated mice (solid columns) compared with control groups (open columns). FIG. 2c is a graph showing cell numbers of different cellular subsets in the spleen and inguinal LNs of control (open columns) and 2A-treated (solid columns) mice at four months of age (n=3). *, P<0.05; **, P<0.01 by student's t test.

FIG. 3 is a graph showing the grade of skin lesions in MRL/lpr mice treated with rat IgG control (open columns) or 2A (solid columns).

FIG. 4a is a graph showing urinary protein levels in MRL/lpr mice treated with 2A (filled circles) or rat IgG (open circles). Urinary protein levels were assessed monthly and graded semi-quantitatively. FIG. 4b is a graph showing the amount and category of inflammation in the kidneys of mice treated with 2A or control IgG as indicated.

FIGS. 5a and 5b are graphs showing the levels of IgG anti-DNA and total IgG, respectively, in MRL/lpr mice treated with 2A (filled circles) or control IgG (open circles). Measurements were taken before initiation of treatment at the age of two months and then monthly for two months. FIG. 5c is a graph showing the ratio of IgG anti-DNA versus total IgG in the mice. FIGS. 5d and 5e are graphs plotting the levels of IgG2a anti-DNA and IgG1 anti-DNA, respectively. FIG. 5f is a graph showing the survival of treated and untreated control mice. In all graphs, n=10; *, P<0.05; **, P<0.01.

FIG. 6a is a series of histograms showing the levels of apoptosis in Thy-1⁺B220⁺ splenocytes cultured in vitro for 0 (left panels) or 6 hours (middle panels) with 2A or control IgG The histograms in the right panels show the levels of apoptosis in CD69 expressing DNTC after treatment with 2A or IgG. FIG. 6b is a graph showing the number of anti-DNA-secreting B cells spleens from B6/lpr mice one week after treatment with 2A. The data are shown as anti-DNA-secreting B cell number per ten thousand B cells. FIG. 6c contains scatter plots produced by flow cytometry, showing the level of IFN-γ production in T cells from B6/lpr mice treated with 2A or control IgG. FIG. 6d is a series of scatter plots produced by flow cytometry, showing the CD11b⁺GR-1⁺ cell population in B6/lpr mice treated with 2A or IgG. All of the above results are representatives of three experiments. FIG. 6e is a graph showing the level of IgG anti-DNA in sera from MRL/lpr mice treated with 2A and/or anti-IFN-γ (n=3).

### DETAILED DESCRIPTION

The invention is based on the discovery that, in a murine model of SLE, treatment with an antibody specific for 4-1BB resulted in decreased lymphadenopathy, decreased autoantibody production, and decreased kidney disease, and to prolonged survival. Beneficial effects were observed whether the animals were treated before or after onset of overt disease symptoms. While the invention is not limited by a particular mechanism, the therapeutic and prophylactic effects of the 4-1BB-specific antibody apparently were mediated by increased apoptosis of DNTC and autoreactive B cells. Thus, the invention provides methods of treatment and/or prophylaxis of autoimmune diseases, lymphoproliferative diseases, and allergies by depleting DNTC as well as autoreactive B cells. Moreover, the invention provides methods for inducing death of DNTC and autoreactive B cells.

4-1BB is a member of the tumor necrosis factor (TNF) receptor superfamily, and is a costimulatory receptor molecule (Vinay and Kwon (1998) Semin. Immunol. 10:481-489; and Kwon et al. (2000) Mol. Cells 10:119-126). 4-1BB is primarily expressed on activated T cells (Pollok et al. (1993) J. Immunol. 150:771-781) and NK cells (Melero et al. Cell. Immunol. 190:167-172). The natural ligand for 4-1BB is 4-1BB ligand (4-1BBL), which has been detected on activated B and T cells, macrophages, and dendritic cells (Goodwin et al. (1993) Eur. J. Immunol. 23:2631-2641; Pollok et al. (1994) Eur. J. Immunol. 24:367-374; and Alderson et al. (1994) Eur. J. Immunol. 24:2219-2227). As described herein, 4-1BB agonists such as 4-1BBL and anti-4-1BB antibodies can be used to stimulate AICD of DNTC and autoreactive B cells.

Polypeptides and Antibodies

The invention provides molecules that bind to 4-1BB. The molecules provided herein can be polypeptides, for example. As used herein, a polypeptide is an amino acid chain, regardless of length or post-translational modification (e.g., phosphorylation or glycosylation). The polypeptides provided herein can bind specifically to 4-1BB, and upon administration to a mammal (e.g., a mouse or a human), can activate an immune response and cause AICD of DNTC and/or autoreactive B cells. Polypeptides of the invention also can lead to AICD of DNTC and autoreactive B cells when incubated in vitro with immune cells. As used herein, a “DNTC” is a T cell that does not express CD4 and CD8.

The molecules provided herein typically are 4-1BB agonists. As used herein, an “agonist” for a particular receptor is a molecule that can interact with the receptor and stimulate its activity. The natural ligand for 4-1BB is 4-1BBL. Other 4-1BB agonists can stimulate 4-1BB activity to produce the same or similar effects as 4-1BBL.

The 4-1BB agonist useful in the methods provided herein can be 4-1BBL or a functional fragment of 4-1BBL, i.e., a fragment of 4-1BBL that binds to 4-1BB with at least 20% (e.g., at least 20%, 30%, 40%, 50%, 60%, 70%, 80%, 90%, 95%, 98%, 99%, 99.5%, or even 100%) of the avidity with which full-length 4-1BBL binds to 4-1BB, and fuinctions to activate the receptor and potentiate an immune response.

Alternatively, a 4-1BB agonist can be an antibody that has specific binding activity for 4-1BB. The terms “antibody” and “antibodies” encompass intact molecules as well as fragments thereof that are capable of binding to 4-1BB. An antibody can be of any immunoglobulin (Ig) class, including IgM, IgA, IgD, IgE, and IgG, and any subclass thereof. Antibodies of the IgM class typically are pentavalent and may be particularly useful because one antibody molecule can cross-link a plurality of 4-1BB polypeptides. Immune complexes containing Ig molecules that are cross-linked (e.g., cross-linked IgG) and are thus multivalent also could be capable of cross-linking a plurality of 4-1BB molecules, and may be particularly useful.

As used herein, an “epitope” is a portion of an antigenic molecule to which an antibody binds. Antigens can present more than one epitope at the same time. For polypeptide antigens, an epitope typically is about four to six amino acids in length. Two different immunoglobulins can have the same epitope specificity if they bind to the same epitope or set of epitopes.

The terms “antibody” and “antibodies” include polyclonal antibodies, monoclonal antibodies, humanized or chimeric antibodies, and antibody fragments such as single chain Fv antibody fragments, Fab fragments, and F(ab)₂ fragments. Polyclonal antibodies are heterogeneous populations of antibody molecules that are specific for a particular antigen, while monoclonal antibodies are homogeneous populations of antibodies to a particular epitope contained within an antigen.

Polyclonal antibodies can be isolated from, for example, the sera of immunized animals. Methods for isolation of polyclonal antibodies include purification from mammalian serum using techniques that include, without limitation, chromatography.

Monoclonal antibodies can be prepared using, for example, standard hybridoma technology. In particular, monoclonal antibodies can be obtained using any technique that provides for the production of antibody molecules by continuous cell lines in culture as described, for example, by Kohler et al. (1975) Nature 256:495-497, the human B-cell hybridoma technique of Kosbor et al. (1983) Immunology Today 4:72, and Cote et al. (1983) Proc. Natl. Acad. Sci. USA 80:2026-2030, and the EBV-hybridoma technique of Cole et al., Monoclonal Antibodies and Cancer Therapy, Alan R. Liss, Inc. pp. 77-96 (1983). A hybridoma producing monoclonal antibodies of the invention can be cultivated in vitro or in vivo. For example, monoclonal antibody 2A was produced in vitro by a hybridoma that was generated by fusing (a) spleen cells from a rat immunized with mouse 4-1BB-Tg, and (b) mouse Sp2/0 myeloma cells (Wilcox et al. (2002) J. Clin. Invest. 109:651-659).

Antibodies that bind to 4-1BB also can be produced by, for example, immunizing host animals (e.g., rabbits, chickens, mice, guinea pigs, or rats) with 4-1BB. A 4-1BB polypeptide or a portion of a 4-1BB polypeptide can be produced recombinantly, by chemical synthesis, or by purification of the native protein, and then used to immunize animals by injection of the polypeptide. Adjuvants can be used to increase the immunological response, depending on the host species. Suitable adjuvants include Freund's adjuvant (complete or incomplete), mineral gels such as aluminum hydroxide, surface-active substances such as lysolecithin, pluronic polyols, polyanions, peptides, oil emulsions, keyhole limpet hemocyanin (KLH), and dinitrophenol. Standard techniques can be used to isolate antibodies generated in response to the 4-1BB immunogen from the sera of the host animals. Such techniques are useful for generating antibodies that have similar characteristics to 2A (e.g., similar epitope specificity and other functional similarities).

Antibodies such as 2A also can be produced recombinantly. The amino acid sequence (e.g., the partial amino acid sequence) of an antibody provided herein can be determined by standard techniques, and a cDNA encoding the antibody or a portion of the antibody can be isolated from the serum of the subject (e.g., the human patient or the immunized host animal) from which the antibody was originally isolated. The cDNA can be cloned into an expression vector using standard techniques. The expression vector then can be transfected into an appropriate host cell (e.g., a Chinese hamster ovary cell, a COS cell, or a hybridoma cell), and the antibody can be expressed and purified.

Antibody fragments that have specific binding affinity for 4-1BB and retain cross-linking function also can be generated by techniques such as those disclosed above. Such antibody fragments include, but are not limited to, F(ab′)₂ fragments that can be produced by pepsin digestion of an antibody molecule, and Fab fragments that can be generated by reducing the disulfide bridges of F(ab′)₂ fragments. Alternatively, Fab expression libraries can be constructed. See, for example, Huse et al. (1989) Science 246:1275-1281. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques, such as those disclosed in U.S. Pat. No. 4,946,778. Such fragments can be rendered multivalent by, for example, biotinylation and cross-linking, thus generating antibody fragments that can cross-link a plurality of 4-1BB molecules.

Nucleic Acids, Vectors, and Host Cells

The invention also provides nucleic acids encoding molecules (e.g., polypeptides and antibodies) that bind specifically to 4-1BB. As used herein, the term “nucleic acid” refers to both RNA and DNA, including cDNA, genomic DNA, and synthetic (e.g., chemically synthesized) DNA. A nucleic acid molecule can be double-stranded or single-stranded (i.e., a sense or an antisense single strand). Nucleic acids of the invention include, for example, cDNAs encoding the light and heavy chains of the 2A monoclonal anti-4-1BB antibody.

An “isolated nucleic acid” refers to a nucleic acid that is separated from other nucleic acid molecules that are present in a vertebrate genome, including nucleic acids that normally flank one or both sides of the nucleic acid in a vertebrate genome. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided that one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

The isolated nucleic acid molecules provided herein can be produced by standard techniques, including, without limitation, common molecular cloning and chemical nucleic acid synthesis techniques. For example, polymerase chain reaction (PCR) techniques can be used to obtain an isolated nucleic acid molecule encoding 2A of a portion of 2A. Isolated nucleic acids of the invention also can be chemically synthesized, either as a single nucleic acid molecule (e.g., using automated DNA synthesis in the 3′ to 5′ direction using phosphoramidite technology) or as a series of polynucleotides. For example, one or more pairs of long polynucleotides (e.g., >100 nucleotides) can be synthesized that contain the desired sequence, with each pair containing a short segment of complementarity (e.g., about 15 nucleotides) such that a duplex is formed when the polynucleotide pair is annealed. DNA polymerase is used to extend the polynucleotides, resulting in a single, double-stranded nucleic acid molecule per polynucleotide pair.

The invention also provides vectors containing nucleic acids such as those described above. As used herein, a “vector” is a replicon, such as a plasmid, phage, or cosmid, into which another DNA segment may be inserted so as to bring about the replication of the inserted segment. The vectors of the invention can be expression vectors. An “expression vector” is a vector that includes one or more expression control sequences, and an “expression control sequence” is a DNA sequence that controls and regulates the transcription and/or translation of another DNA sequence. Similarly, a “transcriptional regulatory element” is an expression control sequence that controls and regulates the transcription of another DNA sequence.

In the expression vectors of the invention, a nucleic acid (e.g., a nucleic acid encoding the light and/or heavy chains of 2A) is operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of a transcriptional regulatory element in a cell when RNA polymerase is able to transcribe the coding sequence into mRNA, which then can be translated into the protein encoded by the coding sequence. Expression vectors provided herein thus are useful to produce 2A, as well as other molecules that bind to an activate 4-1BB.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

An expression vector can include a tag sequence designed to facilitate subsequent manipulation of the expressed nucleic acid sequence (e.g., purification or localization). Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide including at either the carboxyl or amino terminus.

The invention also provides host cells containing vectors of the invention. The term “host cell” is intended to include prokaryotic and eukaryotic cells into which a recombinant expression vector can be introduced. As used herein, “transformed,” “transfected,” and “transduced” encompass the introduction of a nucleic acid molecule (e.g., a vector) into a cell by one of a number of techniques. Although not limited to a particular technique, a number of these techniques are well established within the art. Prokaryotic cells can be transformed with nucleic is acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Suitable methods for transforming and transfecting host cells are found in Sambrook et al., Molecular Cloning: A Laboratory Manual (2^{nd} edition), Cold Spring Harbor Laboratory, New York (1989), and reagents for transformation and/or transfection are commercially available (e.g., Lipofectin® (Invitrogen/Life Technologies); Fugene (Roche, Indianapolis, Ind.); and SuperFect (Qiagen, Valencia, Calif.)).

Methods for Treating and/or Prophylaxis

The invention provides methods for treating or preventing diseases such as autoimmune disorders, hyper-proliferative (e.g., lymphoproliferative) disorders, and allergies. Without being bound by a particular mechanism, the methods provided herein can be used to treat or prevent such diseases by activating an immune response and depleting CD4⁻/CD8⁻ double negative T cells (DNTC) and/or autoreactive B cells. Thus, the invention also provides methods for depleting DNTC and/or autoreactive B cells. The methods provided herein include contacting cells in vitro or in a subject with a 4-1BB-binding agent such as a 4-1BB agonist. In some embodiments, DNTC and/or autoreactive B cells are depleted due to AICD.

As used herein, “depleting” a particular cell type in a subject or in vitro means that the number of cells of a particular type (e.g., DNTC) is reduced after administration of a 4-1BB agonist. Typically, a cell population is depleted by at least 20% (e.g., at least 20%, 25%, 30%, 40%, 50%, 60%, 70%, 80%, 90%, 95%, 99o, or even 100%) after treatment. As used herein, the term “inducing death” of a particular cell means that the cell is dead after treatment with a 4-1BB agonist. The death of a DNTC or an autoreactive B cell may occur through, for example, AICD following administration of a 4-1BB agonist to the cell. A 4-1BB agonist can be administered to such a cell either in vivo or in vitro.

As used herein, “prophylaxis” can mean prevention of the symptoms of a disease, a delay in onset of the symptoms of a disease, or a lessening in the severity of subsequently developed disease symptoms. “Prevention” should mean that symptoms of the disease (e.g., an infection) are essentially absent. As used herein, “therapy” can mean a complete abolishment of the symptoms of a disease or a decrease in the severity of the symptoms of the disease. As used herein, a “protective” immune response is an immune response that is prophylactic and/or therapeutic.

Molecules of the invention typically are administered to a subject or to a cell in an “effective amount.” As used herein, an “effective amount” is an amount of a molecule (e.g., an agonistic anti-4-1BB antibody) to deplete DNTC and/or autoreactive B cells in a subject, or to cause death of a DNTC or an autoreactive B cell either in a subject or in vitro.

Methods for depleting DNTC and/or autoreactive B cells in a subject can include (a) identifying a subject having or at risk for having or developing an autoimmune disorder, a lymphoproliferative disorder, or an allergy; and (b) administering to the subject an effective amount of a 4-1BB binding molecule (e.g., an agonistic anti-4-1BB antibody such as 2A, or a composition containing such an antibody). Methods of the invention also can include steps for identifying a subject in need of such treatment and/or monitoring a treated subject for symptoms. Diseases that can be treated with methods of the invention include, without limitation, SLE, insulin-dependent diabetes mellitus (IDDM), inflammatory bowel disease, a celiac disease, an autoimmune thyroid disease, Sjogren's Syndrome, autoimmune gastritis, pernicious anemia, autoimmune hepatitis, cutaneous autoimmune diseases, autoimmune dilated cardiomyopathy, myocarditis, myasthenia gravis, vasculitis, an autoimmune disease of the eye, an autoimmune disease of the muscle, an autoimmune disease of the testis, an autoimmune disease of the ovary, a hyper-proliferative (e.g., lymphoproliferative) disorder, or an allergy.

SLE is a chronic autoimmune disease with many manifestations. The production of autoantibodies leads to immune complex formation and subsequent deposition in many tissues (e.g., glomeruli, skin, lungs, synovium, and mesothelium). Symptoms of SLE include, for example, rashes, fever, mouth or nose ulcers, joint pain and/or swelling, headache, and muscle aches and/or tenderness. Renal disease is common with SLE because the immune complexes often are deposited in the renal glomeruli. Despite therapy, progression to chronic renal failure is common. In mouse models of SLE, significant proteinuria is observed concomitant with the serological appearance of antibodies to DNA and histones, as well as immune complexes of the IgG1, IgG2a, and IgG2b subclasses. The median survival for such mice is 6 months, and mortality typically results from renal failure. B cells and autoantibodies are thought to play essential roles in disease development, and agents that interfere with autoantibody production have been shown to attenuate the disease.

IDDM is a chronic autoimmune disease characterized by pancreatic beta cell destruction, which manifests as a disturbance of multiple metabolic pathways (Zimmet (1997) Medicine 25:1-3). IDDM affects carbohydrate metabolism, and impaired glucose tolerance (i.e., increased levels of glucose in the blood) is the most apparent effect [Hunter, in Effective Care in Pregnancy and Childbirth, Volume 1. Editors: Chalmers, Enkin, and Keirse. Oxford University Press. pp. 578-593 (1989)]. Other symptoms include excessive thirst, frequent urination, extreme hunger, fatigue, and weight loss. With IDDM there is a severe, abrupt onset of insulin deficiency, as well as a tendency towards ketosis. Subjects with IDDM typically are dependent upon exogenous insulin.

Lymphoproliferative disorders are a heterogeneous group of expanding, monoclonal or oligoclonal, lymphoid neoplasms. Lymphoproliferative disorders include, e.g., autoimmune lymphoproliferative syndrome, agammaglobulinemia, amyloidosis, leukemia, lymphoma, post-transplant lymphoproliferative disorder, sarcoidosis, X-linked lymphoproliferative syndrome, and Waldenstrom macroglobulinemia. They are progressively more common with age. In children, lymphoproliferative disorders occur only in the setting of immune dysfunction. The risk of true malignancy in affected children ranges from 10- to 300-fold higher than the risk in immunocompetent children. Physical symptoms often include adenopathy, splenomegaly, or symptoms attributable to organ infiltration by an expanding lymphoid clone. Because the gastrointestinal tract or lungs may be affected preferentially in certain subtypes, abdominal bloating or pulmonary findings may dominate the physical examination.

Allergies can be immediate or delayed hypersensitivity allergies. They typically are immediate hypersensitivity allergies. Relevant allergens include antigens from a wide variety of sources, e.g., plants, bacteria, insects, and mammals. Plant antigens include, for example, pollen antigens. Pollen antigens can be in pollen of, for example, grasses, birch trees, cedar trees, cypress trees, or ragweed. Bacterial antigens can be from, for example, Staphylococcus aureus. Fungal (including yeast) antigens, e.g., fungal spore antigens, can be from, for example, Aspergillus fumigatus, Alternari, Basidiomycetes, Actinomycetes, Bipolaris spicifera, Drechslera, Excerohilum, the genus Trichophyton, Candida albicans, or Pityrosporiuum ovale. Insect antigens can be from, for example, body parts, blood (e.g., hemoglobin), feces, or saliva of insects including moths, flies, crickets, ants, beetles, cockroaches, mites, spiders, mosquitoes, and fleas. Venom antigens also are of interest, e.g., venom of the fire ant or members of the order Hymenoptera, e.g., honey bees, yellow jackets, wasps, or hornets. The methods of the invention also can be applied to subjects with allergies to mammalian antigens, e.g., antigens in dander or urine from humans, cats, dogs, rats, mice, guinea pigs, gerbils, or rabbits. Additional allergens of interest are well known to those of skill in the art [see, for example, Platts-Mills (Allergens), in Samter's Immunologic Diseases, Fifth Edition, Volume II. Editors: Frank, Austen, Claman, and Unanue. Little, Brown, and Company, Boston, N.Y., Toronto, and London. pp. 1231-1256 (1995), which is incorporated herein by reference in its entirety].

Molecules (e.g., 4-1BB agonists) useful in the methods provided herein can be administered via a number of methods, including methods that are well known in the art. The method of administration typically will depend upon factors such as whether local or systemic treatment is desired and what area is to be treated. Administration can be, for example, topical (e.g., transdermal, sublingual, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, a 4-1BB agonist can be administered by injection or infusion into the cerebrospinal fluid, preferably with one or more agents capable of promoting penetration of the polypeptides across the blood-brain barrier.

In the methods of the invention, a 4-1BB binding polypeptide (e.g., an agonistic anti-4-1BB antibody) can be delivered directly to a subject or to a DNTC. Alternatively, the delivery to a subject or the contacting of a DNTC can include administering to the subject a nucleic acid containing a polynucleotide encoding the polypeptide, the polynucleotide being operably linked to a transcriptional regulatory element. Alternatively, the delivery to a subject or contacting of a DNTC in a subject can involve: (a) providing a cell from the subject; (b) transfecting or transducing the cell, or a progeny of the cell, with a nucleic acid containing a polynucleotide encoding the 4-1BB agonist, wherein the polynucleotide is operably linked to a transcriptional regulatory element; and (c) administering the transfected or transduced cell, or a progeny of the transfected or transduced cell, to the subject. Naturally, where the cell administered to the subject is a progeny of the transfected or transduced cell, such a progeny cell should retain and express the polynucleotide (encoding the 4-1BB agonist) that is contained in the nucleic acid used for transfection or transfection.

Methods of the invention also can include, in addition to administering a 4-1BB agonist, administering interferon-γ and/or an agent (e.g., an antibody) that binds to Gr-1. Gr-1 is a myeloid differentiation antigen expressed on cells of the myeloid lineage, and serves as a marker for granulocyte maturation (Hestdal et al. (1991) J. Immunol. 147:22-28; and Fleming et al. (1993) J. Immunol. 151:2399-2408).

In the methods of the invention, the subject can be a mammalian subject, e.g., a human, a non-human primate, a cow, a horse, a donkey, a mule, a pig, a sheep, a goat, a dog, a cat, a rabbit, a rat, a mouse, a gerbil, a guinea pig, or a hamster. Alternatively, the subject can be a bird such as a chicken or a turkey.

Compositions and Articles of Manufacture

A 4-1BB agonist (e.g., an agonistic anti-4-1BB antibody such as 2A) may be used for the preparation of a medicament for use in any of the methods described herein (e.g., methods for depleting autoreactive cells to treat autoimmune disorders, allergies, and lymphoproliferative disorders). By these methods, antibodies or compositions in accordance with the invention can be administered to a subject (e.g., a human or another mammal) having a disease or disorder (e.g., SLE) that can be alleviated by enhancing an immune response and stimulating AICD of autoreactive B cells and DNTC. Typically, one or more 4-1BB agonists or compositions can be administered to a subject suspected of having a disease or condition associated with an autoimmune response. Alternatively, one or more 4-1BB agonists or compositions can be administered to a DNTC or an autoreactive B cell in vitro.

Compositions of the invention typically contain one or more polypeptides and compounds described herein. A 4-1BB agonist can be in a pharmaceutically acceptable carrier or diluent, and can be administered in amounts and for periods of time that will vary depending upon the nature of the particular disease, its severity, and the subject's overall condition. Typically, the molecule is administered in an effective amount (i.e., an amount that is effective for depleting DNTC and/or autoreactive B cells in a subject, or an amount effective to induce death of a cell contacted by the molecule). The molecules and methods of the invention also can be used prophylactically, e.g., to minimize autoimmunity in a subject at risk for an autoimmune disorder.

The ability of a 4-1BB agonist to deplete DNTC or autoreactive B cells can be assessed by, for example, flow cytometry of cells obtained from a serum sample of a subject treated with the agonist. Alternatively, the ability of a 4-1BB agonist to deplete autoreactive cells can be determined by an enzyme-linked immunosorbent assay (ELISA) of serum from a treated subject. See, e.g., the Examples herein.

Methods for formulating and subsequently administering therapeutic compositions are well known to those skilled in the art. Dosing generally is dependent on the severity and responsiveness of the disease state to be treated, with the course of treatment lasting from several days to several months or longer, or until a cure is effected or a diminution of the disease state is achieved. Persons of ordinary skill in the art routinely determine optimum dosages, dosing methodologies and repetition rates. Optimum dosages can vary depending on the relative potency of individual polypeptides, and can generally be estimated based on EC₅₀ found to be effective in in vitro and/or in vivo animal models. Typically, dosage is from 0.01 μg to 100 g per kg of body weight, and may be given once or more daily, biweekly, weekly, monthly, or even less often. Following successful treatment, it may be desirable to have the patient undergo maintenance therapy to prevent recurrence of the disease state.

The present invention provides pharmaceutical compositions and formulations that include the 4-1BB-binding molecules of the invention. Such molecules therefore can be admixed, encapsulated, conjugated or otherwise associated with other molecules, molecular structures, or mixtures of compounds such as, for example, liposomes, polyethylene glycol, receptor targeted molecules, or oral, rectal, topical or other-formulations, for assisting in uptake, distribution and/or absorption.

A “pharmaceutically acceptable carrier” (also referred to herein as an “excipient”) is a pharmaceutically acceptable solvent, suspending agent, or any other pharmacologically inert vehicle for delivering one or more therapeutic compounds (e.g., agonistic anti-4-1BB antibodies) to a subject. Pharmaceutically acceptable carriers can be liquid or solid, and can be selected with the planned manner of administration in mind so as to provide for the desired bulk, consistency, and other pertinent transport and chemical properties, when combined with one or more of therapeutic compounds and any other components of a given pharmaceutical composition. Typical pharmaceutically acceptable carriers that do not deleteriously react with amino acids include, by way of example and not limitation: water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

The pharmaceutical compositions of the present invention can be administered by a number of methods, depending upon whether local or systemic treatment is desired and upon the area to be treated. As described above, administration can be, for example, topical, pulmonary, oral, or parenteral.

Formulations for topical administration of 4-1BB agonists include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Nasal sprays are particularly useful, and can be administered by, for example, a nebulizer or another nasal spray device. Administration by an inhaler also is particularly useful. Conventional pharmaceutical carriers, aqueous, powder or oily bases, thickeners and the like may be necessary or desirable.

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders.

Compositions and formulations for parenteral, intrathecal or intraventricular administration can include sterile aqueous solutions, which also can contain buffers, diluents and other suitable additives (e.g., penetration enhancers, carrier compounds and other pharmaceutically acceptable carriers).

Pharmaceutical compositions of the present invention include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsions often are biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Liposomes are vesicles that have a membrane formed from a lipophilic material and an aqueous interior that can contain the composition to be delivered. Liposomes can be particularly useful due to their specificity and the duration of action they offer from the standpoint of drug delivery. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin® (Invitrogen/Life Technologies, Carlsbad, Calif.) and Effectene™ (Qiagen, Valencia, Calif.).

Polypeptides of the invention further encompass any pharmaceutically acceptable salts, esters, or salts of such esters, or any other compound that, upon administration to an animal (e.g., a human), is capable of providing (directly or indirectly) the biologically active metabolite or residue thereof. Accordingly, for example, the invention provides pharmaceutically acceptable salts of polypeptides, prodrugs and pharmaceutically acceptable salts of such prodrugs, and other bioequivalents. The term “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the polypeptides of the invention (i.e., salts that retain the desired biological activity of the parent polypeptide without imparting undesired toxicological effects). Examples of pharmaceutically acceptable salts include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); and salts formed with organic acids (e.g., acetic acid, citric acid, oxalic acid, palmitic acid, or fumaric acid).

Pharmaceutical compositions containing the polypeptides of the present invention also can incorporate penetration enhancers that promote the efficient delivery of polypeptides to the skin of animals. Penetration enhancers can enhance the diffusion of both lipophilic and non-lipophilic drugs across cell membranes. Penetration enhancers can be classified as belonging to one of five broad categories, i.e., surfactants (e.g., sodium lauryl sulfate, polyoxyethylene-9-lauryl ether and polyoxyethylene-20-cetyl ether); fatty acids (e.g., oleic acid, lauric acid, myristic acid, palmitic acid, and stearic acid); bile salts (e.g., cholic acid, dehydrocholic acid, and deoxycholic acid); chelating agents (e.g., disodium ethylenediaminetetraacetate, citric acid, and salicylates); and non-chelating non-surfactants (e.g., unsaturated cyclic ureas). Alternatively, inhibitory polypeptides can be delivered via iontophoresis, which involves a transdermal patch with an electrical charge to “drive” the polypeptide through the dermis.

Certain embodiments of the invention provide pharmaceutical compositions containing (a) one or more 4-1BB agonists and (b) one or more other agents that function by a different mechanism. For example, anti-inflammatory drugs, including but not limited to nonsteroidal anti-inflammatory drugs and corticosteroids, and antiviral drugs, including but not limited to ribivirin, vidarabine, acyclovir and ganciclovir, can be included in compositions of the invention. Other non-polypeptide agents (e.g., chemotherapeutic agents) also are within the scope of this invention. Such combined compounds can be used together or sequentially.

Compositions of the present invention additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions of the present invention, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the polypeptide components within the compositions of the present invention. The formulations can be sterilized if desired.

The pharmaceutical formulations of the present invention, which can be presented conveniently in unit dosage form, can be prepared according to conventional techniques well known in the pharmaceutical industry. Such techniques include the step of bringing into association the active ingredient(s) (e.g., an agonistic anti-4-1BB antibody) with the desired pharmaceutical carrier(s) or excipient(s). Typically, the formulations can be prepared by uniformly and bringing the active ingredients into intimate association with liquid carriers or finely divided solid carriers or both, and then, if necessary, shaping the product. Formulations can be sterilized if desired, provided that the method of sterilization does not interfere with the effectiveness of the polypeptide contained in the formulation.

The compositions of the present invention can be formulated into any of many possible dosage forms such as, but not limited to, tablets, capsules, liquid syrups, soft gels, suppositories, and enemas. The compositions of the present invention also can be formulated as suspensions in aqueous, non-aqueous or mixed media. Aqueous suspensions further can contain substances that increase the viscosity of the suspension including, for example, sodium carboxymethylcellulose, sorbitol, and/or dextran. Suspensions also can contain stabilizers.

The 4-1BB agonists provided herein can be combined with packaging material and sold as kits for depleting DNTC and/or autoreactive B cells, and treating or preventing disease. Components and methods for producing articles of manufacture are well known. Articles of manufacture may combine one or more of the 4-1BB agonists set out in the above sections. In addition, the article of manufacture further may include, for example, buffers or other control reagents for depleting or monitoring depletion of DNTC and/or autoreactive B cells. Instructions describing how the agonists are effective for depleting DNTC or treating/preventing disease can be included in such kits.

The invention will be further described in the following examples, which do not limit the scope of the invention described in the claims.

### EXAMPLE 1

Materials and Methods

Mice: B6.MRL-Tnfrsf6^{lpr} (B6/lpr), MRL/MpJ-Tnfrsf6^{lpr} (MRL/lpr), and MRL.129P2 (B6)-Tnfsf6^{tmlqsa} (Fas^{-/-}) mice were purchased from The Jackson Laboratory (Bar Harbor, Me.). C57BL/6 wild type (B6/wt) mice were purchased from the National Cancer Institute (Frederick, Md.).

In vivo treatment with antibodies: 2A, an agonistic monoclonal antibody (mAb) against 4-1BB; was generated as previously described (Wilcox et al., supra). Rat IgG was purchased from Sigma Chemical Co. (St. Louis, Mo.) and served as a control antibody. Starting at two to three months of age, mice were given weekly intraperitoneal (i.p.) injections of 200 μg/mouse 2A or rat IgG, for three weeks.

Flow cytometric analysis: The following antibodies were purchased from BD-PharMingen (San Diego, Calif.): Cy-Chrome™-labeled CD4 (H129.19), R-phycoerythrin (R-PE)-conjugated anti-mouse CD8a (53-6.7), R-PE-conjugated anti-mouse Thy-1.2 (53-2.1), Fluorescein (FITC)-labeled anti-mouse CD45R/B220 (RA3-6B2), FITC-labeled anti-mouse CD69 (H1.2F3), Cy-Chrome™-labeled anti-mouse CD44 (IM7) R-PE-conjugated anti-mouse CD62L (MEL-14), FITC-labeled anti-mouse Gr-1 (RB6-8C5) and biotin-labeled anti-mouse CD11b (M1/70), and FITC-labeled anti-mouse IFN-γ (XMG1.2). R-PE-conjugated streptavidin was obtained from Immunotech (Marseille, France). Cells were double- or triple-stained with the indicated antibodies according to standard procedures, and were analyzed on a FACScan (BD Biosciences, Mountain View, Calif.) using the CellQuest program. Cells were stained with Annexin-V (PharMingen) for detection of apoptosis, according to the manufacturer's protocol. For intracellular IFN-γ staining, single-cell suspensions from spleen were stimulated with 50 ng/ml PMA plus 500 ng/ml ionomycin for 4 hours at 37° C. in the presence of 20 μg/ml brefeldin A. After fixation in 4% formaldehyde, the cells were stained intracellularly for IFN-γ in the presence of 0.5% saponin for cell perrneabilization, followed by staining of cell surface markers. For analysis, all splenocytes were gated in forward vs. side scatter for the entire study, and in certain cases T cell subsets were further gated as mentioned in the relevant figures.

Detection of antibodies by ELISA: Serum samples were collected monthly and examined for the presence of autoantibodies by ELISA. Anti-DNA autoantibody isotypes were examined as follows: Serial serum dilutions starting from 10⁻² were incubated at room temperature for 2 hours on ELISA plates (Dynex Technologies, Inc., Chantilly, Va.) coated with 250 μg/ml herring sperm DNA (Sigma). Thereafter, alkaline phosphatase (AP) conjugated goat anti-mouse IgG(H+L), IgG1, IgG2a, IgG2b, and IgG3 antibodies (Southern Biotechnology Associates, Birmingham, Ala.) were added to the plate. Plates were incubated with p-Nitrophenyl Phosphate substrate (Sigma), and OD (405 nm) was measured by spectrophotometer (Molecular Devices, Sunnyvale, Calif.). For detection of total IgG, goat anti-mouse IgG(H+L) (Southern Biotechnology Associates, Birmingham, Ala.) was used to coat ELISA plates. Experimental values from separate experiments are expressed as mg/ml or are normalized to a single MRL-lpr/lpr-positive control serum used in every assay (arbitrarily defined as 100 U).

Gross pathology: Gross skin pathology was scored monthly. Skin lesions, which consisted of alopecia and scab formation, were scored from 0 to 3 based on the number and area of lesions (0, none; 1, one, <0.5 cm; 2, two or more, <0.5 cm; 3, multiple, >0.5 cm). Lymphadenopathy was evaluated monthly using the number of palpable nodes. Spleen and lymph node enlargement was assessed two months after treatment.

Proteinuria: Urinary protein levels were assessed using reagent strips for urinalysis (Labstix; Bayer Corporation, Elkhart, Ind.). Protein levels were graded semiquantitatively (0, none; 1, 30-100 mg/dl; 2, 100-300 mg/dl; 3, 300-2000 mg/dl; 4, >2000 mg/dl). Each monthly value was determined by sampling and measuring urine on sequential days.

Histopathology: Kidney and skin tissues were collected and immediately immersed in 10% neutral buffered formalin (Fisher, Pittsburgh, Pa.). Formalin-fixed tissue was embedded in paraffin, and 4-em sections were stained with hematoxylin and eosin and evaluated by light microscopy. Kidney samples were blindly examined for pathology at 20× and 40× magnification. Pathology was assessed for the presence of endovasculitis, glomerular crescents, lymphoid hyperplasia, wire loop formation, and mesangial hypercellularity. The glomeruli were evaluated by counting 200 glomerular cross-sections (gcs) per kidney and scoring each glomerulus as: no inflammation, segmental and global involvement of inflammation.

Immunofluorescent evaluation of IgG and C3 depositions in kidney: Kidneys were embedded in OCT compound (Miles Scientific, Naperville, Ill.) and snap frozen at −70° C. Four μm sections were air-dried and fixed with acetone, pretreated with goat serum, and stained with FITC-labeled anti-mouse IgG (Southern Biotechnology Associates, Birmingham, Ala.) and anti-mouse C3 Ab (ICN/Cappel, Aurora, Ohio). Fluorescence was examined by UV-fluorescence microscopy.

Detection of DNA-secreting B cells by ELISPOT: Serial 5-fold dilutions of splenocytes were plated in triplicate into 96-well ELISA spot plates (Cellular Technology, Cleveland, Ohio) pre-coated with 250 μg/ml herring sperm DNA (Sigma). After overnight incubation at 37° C., bound IgG anti-DNA was detected by incubation with AP-conjugated goat anti-mouse IgG (H+L) (Southern Biotechnology Associates) at room temperature for 3 hours. Color development was performed with nitroblue tetrazolium substrate solution (Sigma).

Blockade of IFN-γ and TNF: To block INF-γ, mice were injected i.p. every 4 days with 500 μg rat IgG or anti-IFN-γ (obtained from ascitic fluid collected from RAG-1 knock-out mice inoculated with rat hybridomna XMG1.2). To block TNF, mice received weekly i.p. injections of 300 μg of TNFRI-hIg (kindly provided by Jeff Browning, Biogen, Mass.) for 2 weeks.

Detection of B cell apoptosis induced by IFN-γ activated macrophages: B6/lpr splenocytes (5×10⁵/well) were cultured with or without peritoneal macrophages (1:1) in the presence of different doses of recombinant IFN-γ (PharMingen). The splenocytes were harvested at various time points, and the percentage of B cells undergoing apoptosis was determined by staining with FITC-labeled Annexin V combined with PE-Thy1.2 and Cy-chrome-B220.

Statistics: Student's t test was used to determine the statistical significance of differences between groups. Survival of control and anti-4-1BB treated female MRL/lpr mice was analyzed by the Kaplan-Meier method and the significance of differences was determined by the Log-rand test.

### EXAMPLE 2

Treatment of B6/lpr Mice with Agonistic Anti-4-1BB mAb (2A) Preferentially Activates CD8⁺ T Cells, but Reduces the DNTC and B Cell Populations

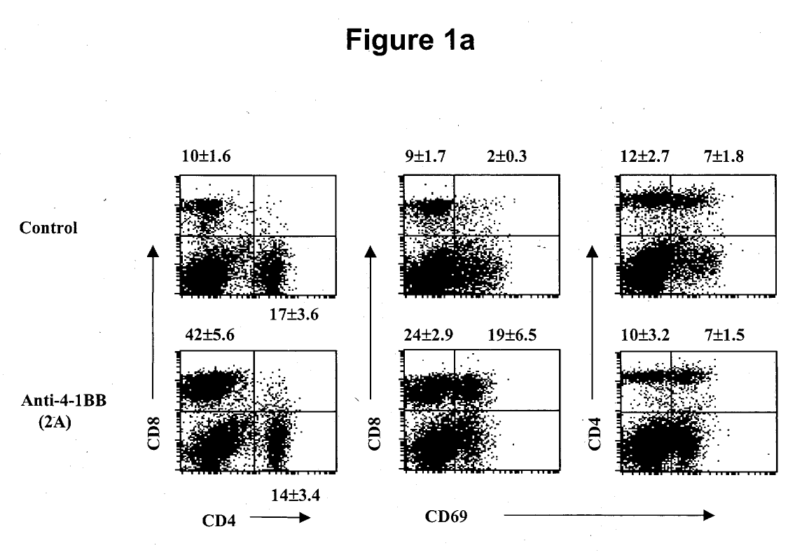

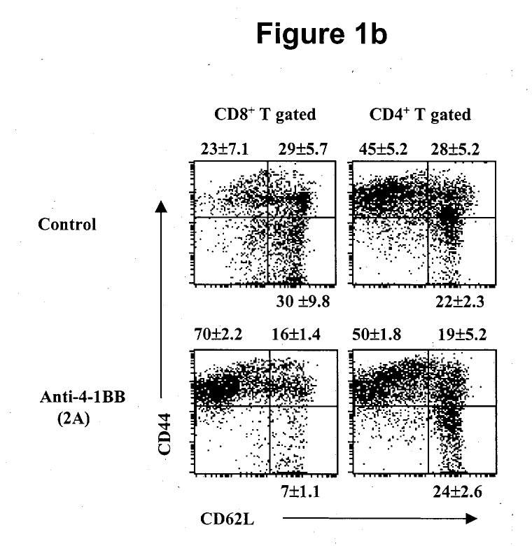

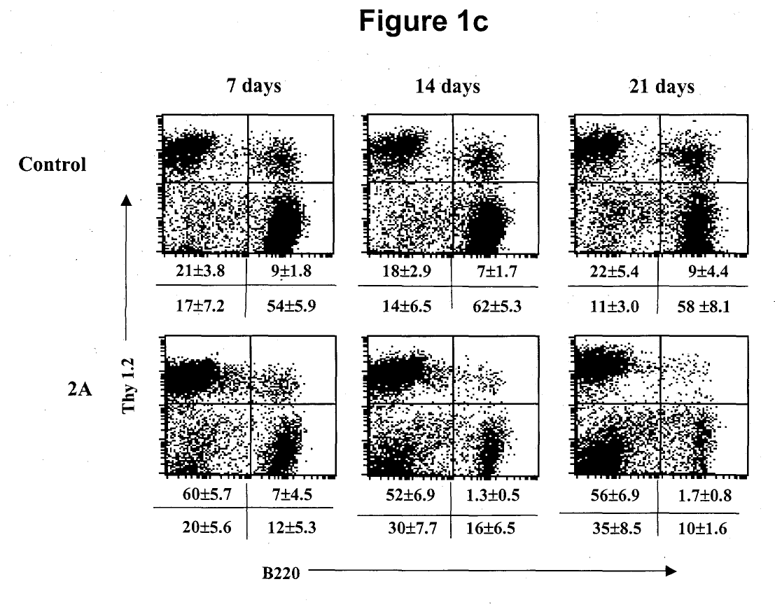

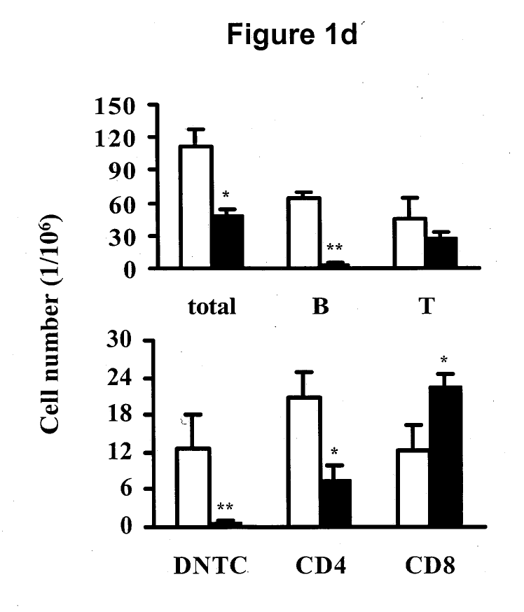

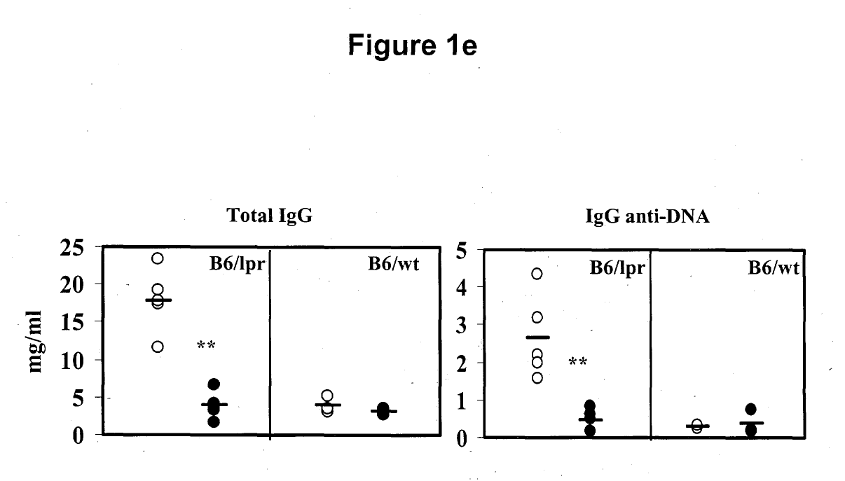

B6/lpr mice are naturally deficient in Fas and suffer from a lymphoproliferative disorder characterized by accumulation of autoreactive lymphocytes soon after birth. To explore the role of 4-1BB signaling in activating autoreactive lymphocytes, two- to three-month-old B6/lpr and B6/wt mice were treated with an agonistic anti-4-1BB mAb (2A) or control rat IgG. The mice were treated at weekly intervals for three weeks, and splenocytes were analyzed by flow cytometry at various time points. By 3 weeks after initiation of treatment, the percentage of CD8⁺ T cells had increased about 3-4 fold in the spleens of 2A-treated mice, whereas CD4⁺ T cell percentages remained the same when compared to control mice (FIG. 1a, left panels). Furthermore, CD4⁺T cell numbers decreased in the 2A-treated mice, while CD8⁺ T cell numbers increased. These changes correlated with up-regulation of the CD69 and CD44 activation markers and down-regulation of CD62L in the CD8+, but not the CD4⁺ T cell subsets (FIG. 1a, right panels and FIG. 1b). These results suggested that 4-1BB signaling preferentially activates CD8⁺ T cells in the absence of Fas signaling.

By 2 to 3 weeks after 2A treatment, the percentages and numbers of splenic DNTC and B cells were dramatically reduced (FIGS. 1c and 1d). The diminished splenic cellularity was due to significant decreases in the B cell, DNTC, and CD4⁺ T cell populations. Sera were collected one week after the final treatment, and IgG anti-DNA and total IgG levels were detected by ELISA. The reduction of the B cell population was accompanied by decreases in production of IgG anti-DNA and total IgC, which were reduced to levels observed in wild type mice (FIG. 1e). Furthermore, the elevated autoantibody levels normally observed in adult B6/lpr mice were not observed 8 weeks after termination of treatment with 2A. A significant reduction of DNTC and B cell percentages also was observed in the lymph nodes, bone marrow, and peripheral blood of treated animals, whereas none of these lymphocytes were detected in various non-lymphoid tissues.

### EXAMPLE 3

Administration of 2A Greatly Ameliorates Lymphadenopathy in MRL/lpr Mice

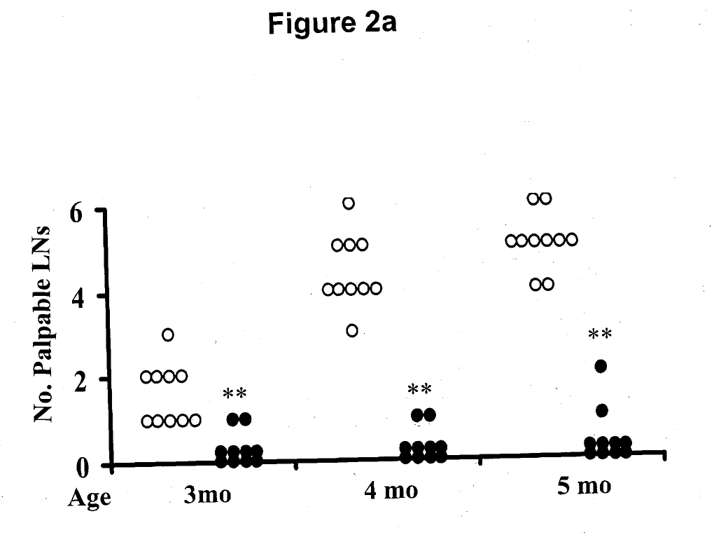

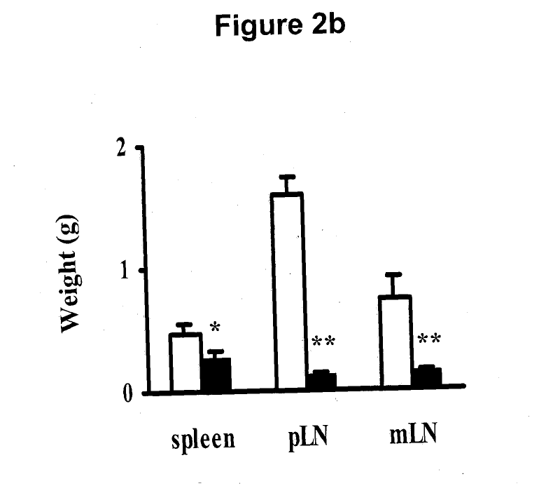

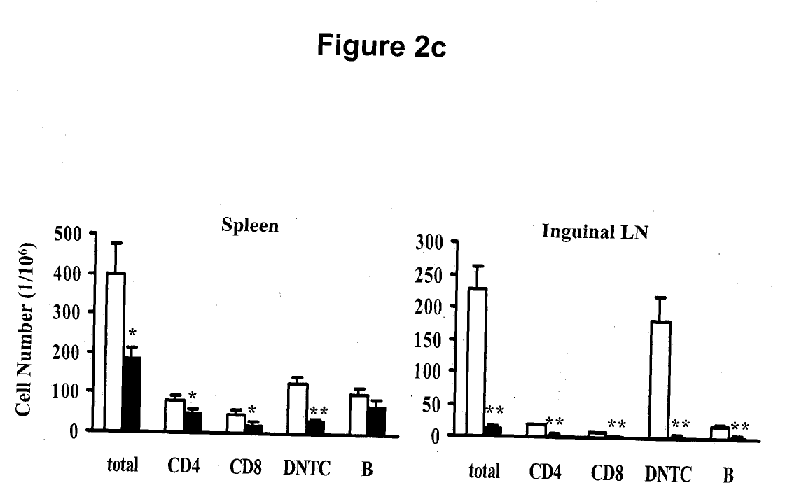

MRL/lpr mice typically exhibit a more severe lymphoproliferative disorder at a younger age than B6/lpr mice, and actually manifest lupus-like features. Nine- to ten-week-old MRL/lpr mice generally have significant numbers of aberrant DNTC and demonstrate higher levels of autoantibody IgG anti-DNA levels in the serum. To test whether 2A has potential therapeutic effects in treating autoimmune diseases, nine- to ten-week-old MRL/lpr mice were treated for three weeks with a weekly dose of 200 μg 2A or control rat IgG. All of the control mice displayed progressively severe lymphadenopathy, whereas only two out of the ten mice in the 2A-treated group developed 1-2 small palpable lymph nodes (LNs) by five months of age (FIG. 2a). In addition, at four months of age the 2A-treated mice had considerably smaller spleens and peripheral LNs than the control mice (FIG. 2b). Lymphocyte numbers and total cell numbers were significantly reduced in the spleen and in the peripheral lymph nodes of 2A-treated mice (FIG. 2c). The sharpest decline was in the number of DNTC, which are a key component of lymphadenopathy in MRL/lpr mice. These results suggest that an agonistic mAb against 4-1BB may stimulate activated lymphocytes, thereby leading to AICD in a Fas-independent manner.

### EXAMPLE 4

2A Treatment Prevents the Development of Skin Lesions in MRL/lpr Mice

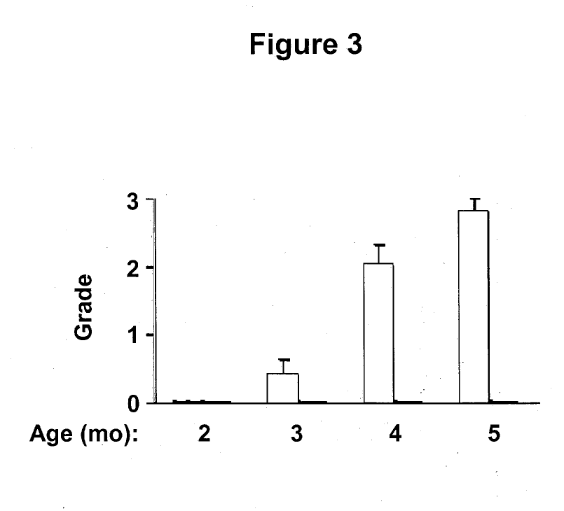

MRL/lpr mice typically develop a progressive spontaneous cutaneous disease, and by five months of age virtually all MRL/lpr mice have large plaque-like cutaneous lesions on the posterior neck. To evaluate the effect of 2A on this cutaneous disease, nine- to ten-week-old female MRL/lpr mice were treated with 2A or control rat IgG three times at weekly intervals. Treatment with 2A completely prevented gross pathologic skin lesions in the entire group, as no cutaneous lesions were detected in any of the 2A-treated mice (FIG. 3). Histological sections of skin (posterior neck) from control mice revealed significant epidermal acanthosis, along with marked dermal chronic inflammatory cell infiltrates. Similar sections from 2A-treated mice exhibited normal architecture and morphology. Thus, the 2A treatment protocol was effective in treating cutaneous lupus-like lesions in MRL/lpr mice.

### EXAMPLE 5

2A Treatment Attenuates Renal Disease in MRL/lpr Mice

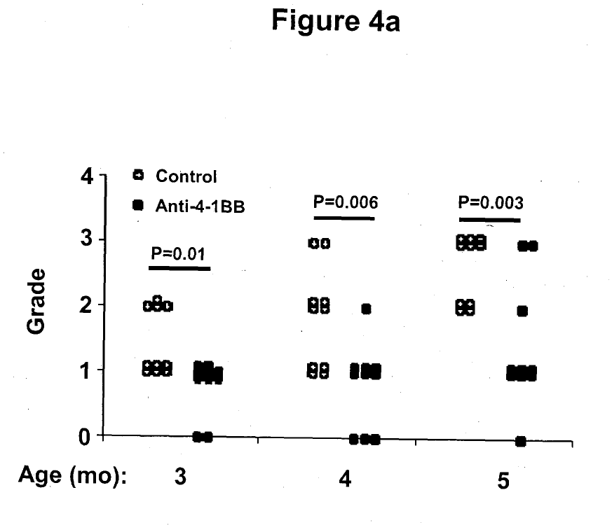

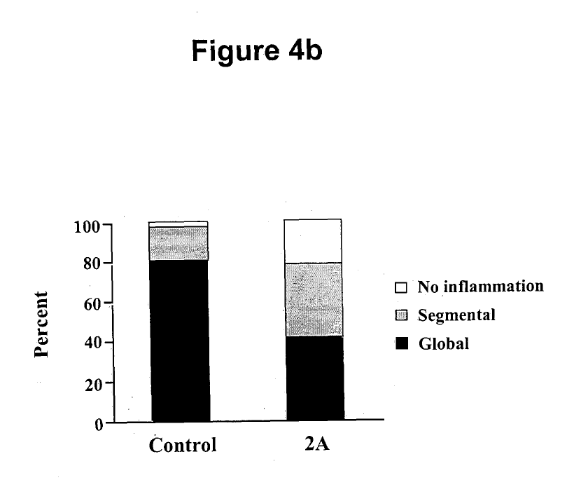

Kidney diseases are considered to be the primary cause of mortality in those afflicted with lupus. The effect of 2A treatment on kidney function in MRL/lpr mice was examined by determining monthly proteinuria levels. Female MRL/lpr mice were treated as described above with 2A or control IgG. Urinary protein levels were assessed monthly using reagent strips for unnalysis and graded semi-quantitatively as described in Example 1. Proteinuria was significantly reduced in the treated mice (FIG. 4a). At five months of age, kidneys were collected and fixed in formalin, and sections were stained with hematoxylin and eosin. Kidney sections from four mice per group were scored for glomerulonephritis, with results classified as no inflammation, segmental inflammation, or global inflammation. Kidney pathology in control mice treated with rat IgG demonstrated severe diffuse global proliferative glomerulonephritis, involving over 80% of total glomeruli, and most of the remaining glomeruli had segmental glomerulonephnitis (FIG. 4b). Histological sections from control mice exhibited prominent perivascular inflammatory cell infiltrate consisting predominantly of lymphocytes and plasma cells, as well as intra- and extra-capillary necrotizing and sclerosing lesions in most glomeruli. In contrast, kidney sections from 2A-treated mice primarily manifested focal proliferative glomerulonephritis, with about 40% segmental involvement and less than 40% global involvement. More than 20% of glomeruli in 2A-treated mice appeared completely normal. Patchy perivascular infiltrate was detected, but to a much lesser degree than was observed in control mice (FIG. 4b).

Lupus models are characterized by direct autoantibody-mediated tissue injury and the deposition of complement-fixing immune complexes. The deposition of complement C3 in the kidney is a key pathologic finding in lupus nephritis (Passwell et al. (1988) J. Clin. Invest. 82:1676-1684). The kidneys of 2A-treated and control mice were stained with FITC labeled goat-anti-mouse IgG or complement C3 and examined for IgG and complement C3 depositions. These studies revealed that both IgG and complement C3 depositions were significantly reduced in 2A-treated mice.

### EXAMPLE 6

2A Treatment Significantly Reduces Autoantibody Production and Prolongs the Survival of MRL/lpr Mice

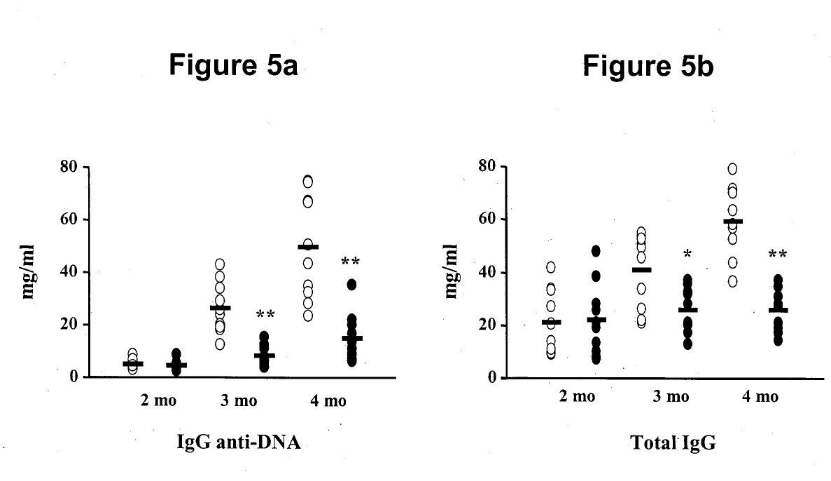

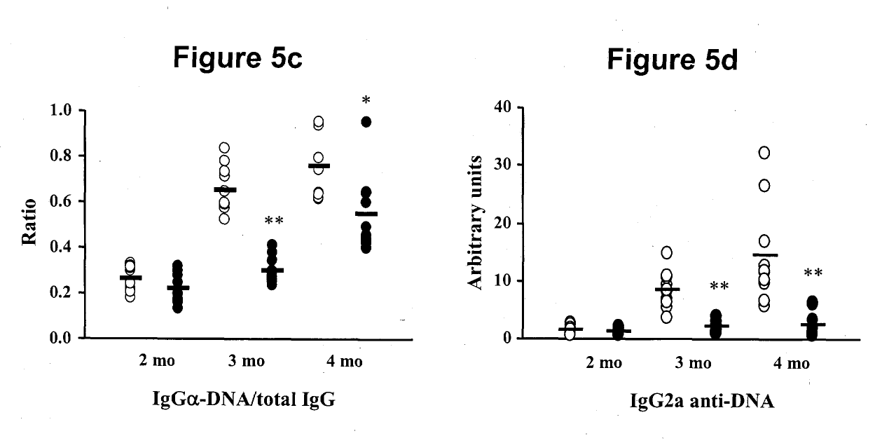

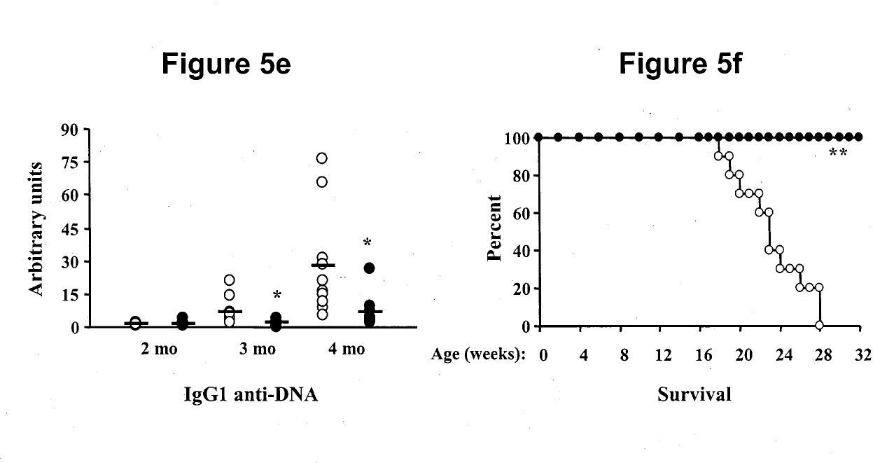

Since autoantibodies are a hallmark of SLE (Cohen and Eisenberg (1991) Annu. Rev. Immunol. 9:243-269; and Hoffman (2001) Front. Biosci. 6:D1369-1378), the effects of 2A treatment on autoantibody production in MRL/lpr mice were examined. Mice were treated with 2A or control rat IgG as described above. Sera were collected before the treatment at the age of two months and then monthly after treatment initiation, and total IgG and autoantibody levels were detected by ELISA. Treatment with 2A significantly decreased autoantibody IgG anti-DNA levels (FIG. 5a), and to a lesser extent, decreased total IgG production (FIG. 5b). The ratios of IgG anti-DNA versus total IgG levels in MRL/lpr mice also were reduced (FIG. 5c). An increase in IgG2a isotype levels is associated with disease pathogenesis in lpr models (Jacobson et al. (1997) Immunol. Rev. 156:103-110). Treatment with 2A greatly reduced the levels of the IgG2a and IgG1 anti-DNA isotypes (FIGS. 5d and 5e), but not the levels of the IgG2b and IgG3 isotypes.

Strikingly, 2A treatment also significantly prolonged the survival of MRL/lpr mice (FIG. 5f). Most of the control mice died by 24 weeks, whereas 2A-treated mice remained healthy for another two months, at which point the experiments were terminated. Thus, these data indicate that an agonistic antibody against 4-1BB can be a powerful clinical agent for treating spontaneous autoimmune diseases and prolonging survival. The most important criterion for determining the clinical relevance of an immunotherapeutic protocol for spontaneous autoimmune diseases is whether the treatment can prevent or delay the progression of a well-established and clinically detectable autoimmune disease. To test this criterion, 3 month-old MRL/lpr mice with 1 to 2 palpable LNs and skin lesions were treated for three weeks with a weekly dose of 200 μg 2A or control rat IgG The 2A treatment regimen reduced autoantibody IgG anti-DNA levels and slowed the progression of lymphadenopathy. These results suggest that treatment with 2A could have potential relevance in a clinical setting.

### EXAMPLE 7

2A Treatment Induces Depletion of Activated T and B Cells by Fas- and TNFR-Independent Apoptosis Mechanisms

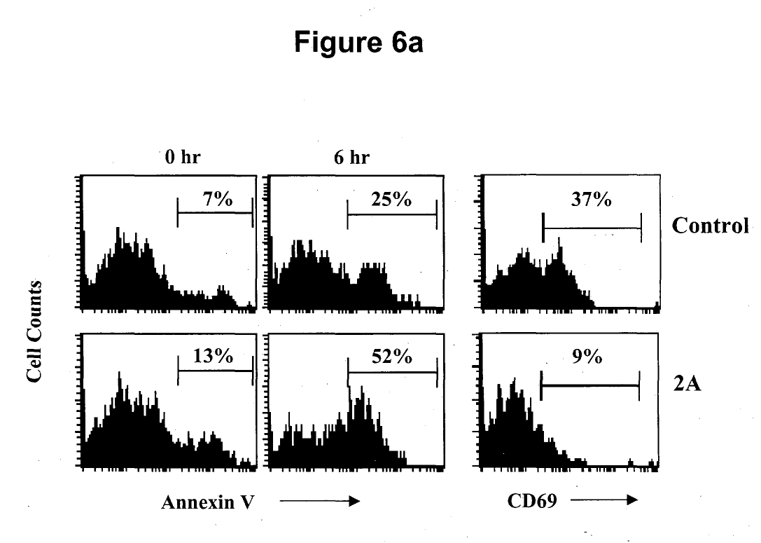

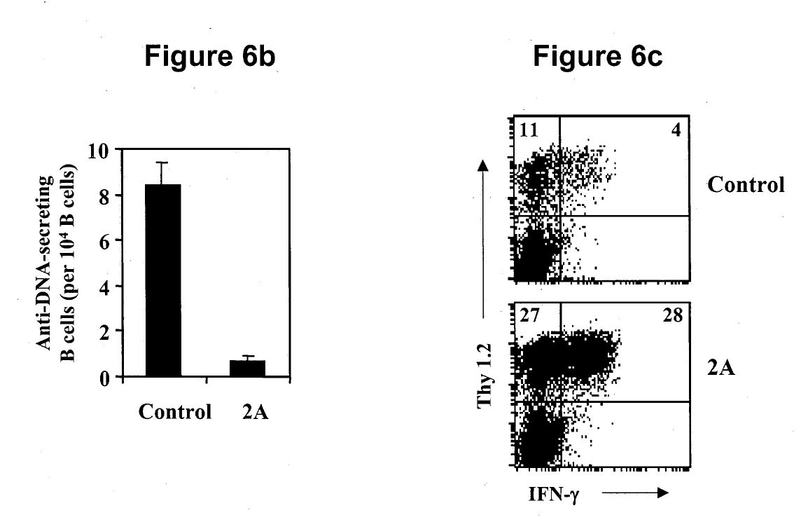

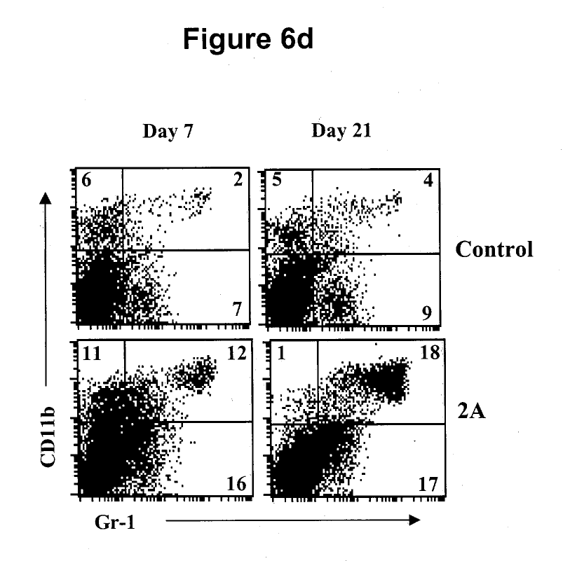

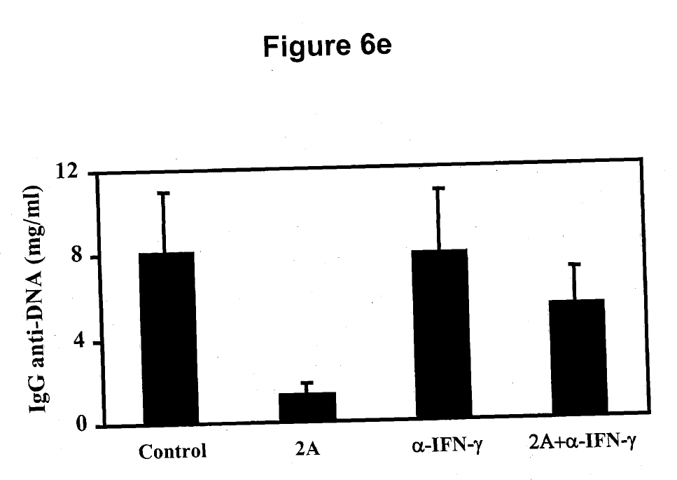

To study the mechanisms that mediate the reduction in DNTC and B cell populations in secondary lymphoid organs following 2A treatment, the fate of these cells was evaluated to determine whether they underwent redistribution or apoptosis. Analyses of DNTC and B cells in the lymph node, bone marrow, and peripheral blood showed similar patterns in each tissue. DNTC and B cells were not detected in various non-lymphoid tissues. These results suggested that the decreased numbers of lymphocytes in the secondary lymphoid tissues is likely due to their depletion as a consequence of 2A treatment. Female mice were treated with 200 μg 2A or control IgG. Five to seven days after treatment, splenocytes were cultured in vitro for 0 or 6 hours and then stained with anti-Thy-1 and anti-B220 combined with Annexin V. A consistent increase in the percentages of apoptotic DNTC was detected in 2A-treated mice (FIG. 6a, left panels, 0 hours). When the cells were cultured for 6 hours, DNTC from the spleen of 2A-treated mice showed increased apoptosis as compared to control cells (FIG. 6a, middle panels). Two weeks after treatment, splenocytes were stained with anti-Thy-1 and anti-B220 combined with anti-CD69. DNTC expressing CD69 were preferentially deleted when compared with CD69 negative cells (FIG. 6a, right panels). In addition, an ˜80% increase in the percentage of apoptotic B cells was detected by Annexin V staining five days after treatment in 2A-treated mice (15±3.7%) versus in control mice (8±1.7%). One week after B6/lpr mice were treated with 2A, splenocytes were collected for ELISPOT assays, which showed that the number of anti-DNA-secreting B cells was greatly reduced by 2A treatment (FIG. 6b).

Signaling via Fas and TNFR can induce apoptosis. Since lpr mice have weak transcriptional expression of Fas antigens (Adachi et al. (1993) Proc. Natl. Acad. Sci. USA 90:1756-1760; and Suda and Nagata (1997) J. Allergy Clin. Immunol. 100:S97-101), a strong 4-1BB costimulatory signal may promote Fas expression on the surface of lymphocytes and induce apoptosis. To test this possibility, Fas-deficient mice were treated with 2A. These studies gave similar results as those obtained with lpr mice, with both the B cell and DNTC populations significantly diminished. By administering TNFR-Ig (300 μg/mouse) weekly in combination with 2A, it was determined that TNF blockade also did not affect the depletion of B cell and DNTC populations in 2A-treated lpr mice. These data suggest that lymphocyte apoptosis induced by 2A treatment was Fas- and TNFR-independent.

### EXAMPLE 8

Reduction of Autoreactive B Cells Mediated by 2A Treatment is IFN-γ-Dependent

To evaluate whether the decrease in autoreactive B cells is IFN-γ-dependent, B6/lpr mice were treated with control IgG or anti-4-1BB. One week later, splenocytes were stained with Thy-1.2 combined with intracellular staining for IFN-γ, and then analyzed by flow cytometry. These experiments revealed that IFN-γ could be responsible for the diminished number of autoreactive B cells, because 2A treatment significantly increased the number of IFN-γ-producing cells (FIG. 6c), including CD4⁺ and CD8⁺ T cells and DNTC. In addition, much higher levels of IFN-γ were detected in 2A-treated MRL/lpr mice than in control mice one week after treatment. Since IFN-γ can activate macrophages, which in turn can potentially apoptose activated lymphocytes (Ding et al. (1988) J. Immunol. 141:2407-2412; Williams et al. (1998) J. Immunol. 161:6526-653 1; and Haendeler et al. (1999) Vitam. Horm. 57:49-77), the effects of 2A treatment on CD11b⁺Gr-1⁺ macrophage/granulocyte populations were examined in B6/lpr mice. A significant increase in the percentage and numbers of these cells was observed in the spleen after treatment with 2A (FIG. 6d).

To test whether B cell depletion was IFN-γ-dependent, mice were treated with anti-IFN-γ in combination with 2A. The combinatorial treatment reversed the effects of treating B6/lpr mice with 2A alone, such that macrophage/granulocyte expansion was decreased and B cell percentages were increased. Anti-IFN-γ treatment alone showed no effect. This result suggested that depletion of autoreactive B cells by 2A treatment is IFN-γ-dependent. In accordance with this finding, the combined treatment also reversed the reduction of autoantibody IgG anti-DNA levels that was initially observed when MRL/lpr mice were treated with 2A alone (FIG. 6e). When 2A treatment was combined with anti-GR-1 administration, a greater expansion of CD11b⁺GR-1⁺ cells was observed, accompanied by a significantly more dramatic reduction of the B population. These results implicate a role for CD11b⁺GR-1⁺ cells in mediating B cell depletion. To directly test this hypothesis, in vitro experiments were performed to confirm that B cell apoptosis was induced by IFN-γ activated macrophages. Splenocytes from B6/lpr mice were cultured with or without peritoneal macrophages in the absence or presence of varying doses of IFN-γ. At 18 and 40 hours, splenocytes were harvested for detection of apoptosis by staining with FITC-labeled Annexin V and cell surface makers. These experiments demonstrated that in the presence of IFN-γ, macrophages greatly enhanced B cell apoptosis (Table 1). In the absence of macrophages, however, increasing the dose of IFN-γ alone did not augment B cell apoptosis.

TABLE 1
Percent apoptotic B cells

| | 18 hours | 40 hours |
|---|---|---|
| No IFN-γ | 6.6 | 9.4 |
| 2.5 ng/ml IFN-γ | 23.9 | 56.2 |
| 25 ng/ml IFN-γ | 40.9 | 79.6 |

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for depleting double negative T cells in a subject, said method comprising:
(a) identifying a subject as having, or at risk of having, an autoimmune disease, a lymphoproliferative disease, or an allergy; and
(b) administering to said subject an effective amount of a 4-1BB agonist.

2. The method of claim 1, wherein said subject is a human.

3. The method of claim 1, further comprising depleting autoreactive B cells in said subject, wherein said 4-1BB agonist is effective to deplete said autoreactive B cells.

4. The method of claim 1, wherein said 4-1BB agonist is an antibody that binds to 4-1BB.

5. The method of claim 4, wherein said antibody is a monoclonal antibody.

6. The method of claim 4, wherein said antibody is 2A.

7. The method of claim 1, further comprising administering interferon-K to said subject.

8. The method of claim 1, further comprising administering a Gr-1-binding agent to said subject.

9. The method of claim 8, wherein said Gr-1-binding agent is an antibody that binds to Gr-1.

10. The method of claim 1, wherein said autoimmune disease or said lymphoproliferative disease is systemic lupus erythematosus.

11. The method of claim 1, wherein said autoimmune disease is insulin-dependent diabetes mellitus.

12. The method of claim 1, wherein said autoimmune disease or said lymphoproliferative disease is selected from the group consisting of an inflammatory bowel disease, a celiac disease, an autoimmune thyroid disease, Sjogren's Syndrome, autoimmune gastritis, pernicious anemia, autoimmune hepatitis, cutaneous autoimmune diseases, autoimmune dilated cardiomyopathy, myocarditis, myasthenia gravis, vasculitis, autoimmune diseases of the muscle, autoimmune diseases of the testis, autoimmune diseases of the ovary, and autoimmune diseases of the eye.

13. The method of claim 1, wherein said allergy is to pollen antigens, fungal antigens, insect antigens, bacterial antigens, mammalian antigens, or insect venom antigens.

14. The method of claim 1, wherein said 4-1BB-binding agent is 4-1BB ligand or a fragment thereof.

15. The method of claim 1, wherein said administering comprises delivering to said subject a nucleic acid comprising a polynucleotide encoding said 4-1BB agonist, wherein said polynucleotide is operably linked to a transcriptional regulatory element.

16. The method of claim 1, wherein said administering comprises:
(i) providing a cell from said subject;
(ii) transfecting or transducing said cell, or a progeny of said cell, with a nucleic acid comprising a polynucleotide encoding said 4-1BB-agonist, wherein said polynucleotide is operably linked to a transcriptional regulatory element; and
(iii) administering said transfected or transduced cell, or a progeny of said transfected or transduced cell, to said subject.

17. The method of claim 1, further comprising:
(c) monitoring said subject for symptoms of said autoimmune disease, lymphoproliferative disease, or allergy.

18. A method for inducing death of a double negative T cell, said method comprising contacting said double negative T cell with an effective amount of a 4-1BB agonist.

19. The method of claim 18, wherein said 4-1BB agonist is an antibody that binds to 4-1BB.

20. The method of claim 19, wherein said antibody is a monoclonal antibody.

21. The method of claim 19, wherein said antibody is 2A.

22. The method of claim 18, wherein said 4-1BB agonist is 4-1BB ligand or a fragment thereof.

23. The method of claim 18, further comprising inducing death-of an autoreactive B cell, wherein said autoreactive B cell is contacted with said effective amount of said 4-1BB agonist.

24. The method of claim 18, wherein said double negative T cell is in vitro.

25. The method of claim 18, wherein said double negative T cell is in a subject.

26. The method of claim 25, wherein said subject is a human.

27. The method of claim 25, wherein said subject has or is at risk for having an autoimmune disease, a lymphoproliferative disease, or an allergy.

28. The method of claim 27, wherein said autoimmune disease or said lymphoproliferative disease is systemic lupus erythematosus.

29. The method of claim 27, wherein said autoimmune disease is insulin-dependent diabetes mellitus.

30. The method of claim 27, wherein said autoimmune disease or said lymphoproliferative disease is selected from the group consisting of an inflammatory bowel disease, a celiac disease, an autoimmune thyroid disease, Sjogren's Syndrome, autoimmune gastritis, pernicious anemia, autoimmune hepatitis, cutaneous autoimmune diseases, autoimmune dilated cardiomyopathy, myocarditis, myasthenia gravis, vasculitis, autoimmune diseases of the muscle, autoimmune diseases of the testis, autoimmune-diseases of the ovary, and autoimmune diseases of the eye.

31. The method of claim 27, wherein said allergy is to pollen antigens, fungal antigens, insect antigens, bacterial allergens, mammalian antigens, or insect venom antigens.

32. The method of claim 25, wherein said contacting comprises administering to said subject said 4-1BB agonist.

33. The method of claim 25, wherein said contacting comprises administering to said subject a nucleic acid comprising a polynucleotide encoding said 4-1BB agonist, wherein said polynucleotide is operably linked to a transcriptional regulatory element.

34. The method of claim 25, wherein said contacting comprises:
(a) providing a cell from said subject;
(b) transfecting or transducing said cell, or a progeny cell of the cell, with a nucleic acid comprising a polynucleotide encoding said 4-1BB agonist, wherein said polynucleotide is operably linked to a transcriptional regulatory element; and
(c) administering said transfected or transduced cell, or a progeny of said transfected or transduced cell, to said subject.
